(19) United States
(12) Patent Application Publication
(10) Pub. No.: US 2021/0299416 A1
(43) Pub. Date: Sep. 30, 2021

(54) BALLOON CANNULA INCLUDING A PLURALITY OF BALLOONS

(71) Applicant: Covidien LP, Mansfield, MA (US)

(72) Inventors: Matthew A. Dinino, Newington, CT (US); Jacob C. Baril, Norwalk, CT (US); Nicolette R. LaPierre, Windsor Locks, CT (US); Saumya Banerjee, Hamden, CT (US)

(21) Appl. No.: 16/828,378

(22) Filed: Mar. 24, 2020

(51) Int. Cl. A61M 25/10; A61M 25/00

(52) U.S. Cl. CPC A61M 25/1011; A61M 2025/1061; A61M 25/10181; A61M 25/0067

(57) ABSTRACT

A balloon cannula includes a housing defining a cavity, a cannula member extending from the housing and defining a lumen in communication with the cavity of the housing, an expandable balloon assembly disposed along the cannula member, and a fluid port adapted to be coupled to a fluid source. The expandable balloon assembly includes a plurality of balloons in a superposed relation. Each balloon of the plurality of balloons is in fluid communication with an adjacent balloon of the plurality of balloons. The fluid port is in fluid communication with the expandable balloon assembly.

### FIELD

The disclosure relates generally to surgical instruments, and more particularly, to a balloon cannula including a plurality of balloons extending along a length thereof.

### BACKGROUND

Minimally invasive surgical procedures, including endoscopic, laparoscopic and arthroscopic procedures, have been used for introducing surgical instruments inside a patient and for viewing portions of the patient's anatomy. Forming a relatively small diameter, temporary pathway to the surgical site is a key feature of most minimally invasive surgical procedures. The most common method of providing such a pathway is by inserting a trocar cannula assembly, through the skin. The trocar cannula assembly may include an expandable balloon configured to enhance securement of the trocar cannula assembly to an opening in tissue.

Obturators are typically designed with a tip that may be used to form an opening through the abdominal wall. An obturator is inserted into a cannula, and then the combined obturator and cannula are together placed against the skin to be penetrated. In order to penetrate the skin, the distal end of the obturator engages the skin, which may or may not have been previously cut with a scalpel. The obturator is then used to penetrate the skin and access the body cavity. By applying pressure against the proximal end of the obturator, the tip of the obturator is forced though the skin and the underlying tissue layers until the cannula and obturator enter the body cavity. The obturator is then withdrawn. The cannula remains in place within the incision for use during the minimally invasive procedure.

It is desirable for the trocar cannula assembly to provide an effective seal against tissue in order to maintain insufflated working space. However, it may be challenging to maintain a seal due to difference in anatomical structure of each patient, such as e.g., abdominal wall thickness. Accordingly, there is a need for a trocar cannula assembly that effectively and safely maintains a fluid-tight seal against tissue.

### SUMMARY

In accordance with the disclosure, a balloon cannula includes a housing defining a cavity, a cannula member extending from the housing and defining a lumen in communication with the cavity of the housing, an expandable balloon assembly disposed along the cannula member, and a fluid port adapted to be coupled to a fluid source. The expandable balloon assembly includes a plurality of balloons in a superposed relation. Each balloon of the plurality of balloons is in fluid communication with an adjacent balloon of the plurality of balloons. The fluid port is in fluid communication with the expandable balloon assembly.

In an aspect, the expandable balloon assembly may be coaxially mounted about the cannula member.

In another aspect, the expandable balloon assembly may be formed of a compressible material.

In yet another aspect, the expandable balloon assembly may be integrally or monolithically formed.

In an aspect, the expandable balloon assembly may have a uniform diameter.

In another aspect, the expandable balloon assembly may have a uniform thickness.

In yet another aspect, each balloon of the plurality of balloons may be configured to expand radially outwards.

In still yet another aspect, each balloon of the plurality of balloons may have a toroidal shape.

In still yet another aspect, first and second balloons of the plurality of balloons may have different thicknesses.

In an aspect, first and second balloons may be radially compressible.

In another aspect, the plurality of the balloons may extend proximally from a distal portion of the cannula member.

In yet another aspect, the cannula member may have a tapered distal end.

In still yet another aspect, the cannula member may be formed of a translucent material.

In an aspect, the expandable balloon assembly may be formed of an elastomer.

In another aspect, the expandable balloon assembly may extend between proximal and distal ends of the cannula member.

In accordance with another aspect of the disclosure, a balloon cannula includes a cannula member defining a lumen dimensioned to receive a surgical object therethrough, an expandable balloon assembly mounted about the cannula member, and a fluid port adapted to be coupled to a fluid source. The expandable balloon assembly is configured to engage tissue in a sealing relation. The expandable balloon assembly includes a plurality of balloons axially arranged between proximal and distal ends of the cannula member. The fluid port is in fluid communication with the expandable balloon assembly.

In an aspect, each balloon of the plurality of balloons may be expandable radially outwards.

In another aspect, each balloon of the plurality of balloons may be formed of a resilient material.

### BRIEF DESCRIPTION OF DRAWINGS

A balloon cannula is disclosed herein with reference to the drawings, wherein:

FIG. 1 is a side view of a balloon cannula in accordance with the disclosure; and

FIG. 2 is a side view of the balloon cannula of FIG. 1, illustrating use thereof.

### DETAILED DESCRIPTION

A balloon cannula is described in detail with reference to the drawings, wherein like reference numerals designate corresponding elements in each of the several views. As used herein, the term “distal” refers to that portion of the instrument, or component thereof which is farther from the user while the term “proximal” refers to that portion of the instrument or component thereof which is closer to the user. In addition, the terms parallel and perpendicular are understood to include relative configurations that are substantially parallel and substantially perpendicular up to about + or −10 degrees from true parallel and true perpendicular. Further, to the extent consistent, any or all of the aspects detailed herein may be used in conjunction with any or all of the other aspects detailed herein.

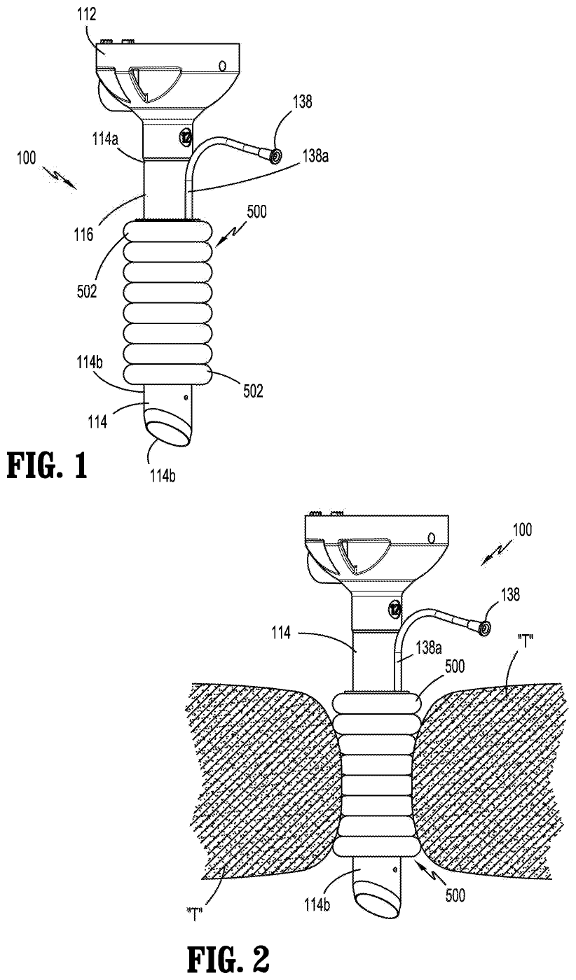

With reference to FIG. 1, an expandable balloon assembly for use with surgical instruments, in the form of a balloon cannula 100 is shown generally as 500. The expandable balloon assembly 500 is configured to secure the balloon cannula 100 to an opening in tissue such that the balloon cannula 100 forms a fluid-tight seal against adjacent tissue. The expandable balloon assembly 500 enables a single balloon cannula 100 to be used for different tissue thicknesses, as will be discussed below.

With continued reference to FIG. 1, the balloon cannula 100 is configured to permit access to an insufflated abdominal cavity during a laparoscopic procedure to permit the introduction of a surgical object for performing various surgical tasks on internal organs within the abdominal cavity. The abdominal cavity is insufflated with fluid (e.g., CO₂), to expand the body wall and provide a working space for the laparoscopic procedure. The surgical object may be a surgical instrument such as laparoscopic or endoscopic clip appliers, obturators, graspers, dissectors, retractors, staplers, laser probes, photographic devices, tubes, endoscopes and laparoscopes, electro-surgical devices and the like. The balloon cannula 100 generally includes a cannula housing 112, a cannula member 114 extending from the cannula housing 112, and an expandable balloon assembly 500 mounted on the cannula member 114. The balloon cannula 100 may optionally include a locking collar (not shown) positioned about a proximal end of the balloon cannula 100 and advanceable to engage the exterior surface of tissue (e.g., the abdominal wall). The locking collar in combination with the expandable balloon assembly 500 may minimize movement of the cannula member 114 as a surgical instrument is either inserted or withdrawn through the balloon cannula 100 and also assists in maintaining a seal about the passage in the abdominal wall.

With continued reference to FIG. 1, the cannula housing 112 is dimensioned for engagement by the clinician and may include one or more internal seals (not shown) adapted to establish a seal about a surgical object introduced therethrough. The internal seals may include, e.g., a duck bill or zero-closure seal positioned in the cannula housing 112. For example, the zero-closure seal may be formed of a suitable resilient material (e.g., silicone) and be configured to inhibit fluids from exiting proximally through the cannula housing 112 in the absence of a surgical object inserted therethrough. The cannula housing 112 and the cannula member 114 may be integrally formed as a single construct. Alternatively, the cannula housing 112 and the cannula member 114 may be monolithically formed. For example, the cannula housing 112 and the cannula member 114 may be formed of suitable biocompatible materials such as medical grade metals (e.g., stainless steel), polymeric materials (e.g., polycarbonate), or combinations thereof. The cannula member 114 may have a tapered distal end 114a.

The expandable balloon assembly 500 is coupled to the cannula member 114 using known techniques such as RF welding, ultrasonic welding, adhesives, etc. The cannula member 114 further includes a fluid port 138 positioned adjacent the cannula housing 112. The fluid port 138 is in fluid communication with the expandable balloon assembly 500 via a supply line 138a. The fluid port 138 is adapted to be coupled to a source of inflation fluids to inflate the expandable balloon assembly 500. For example, the fluid port 138 may include a luer connector for connecting to the source of inflation fluids.

The expandable balloon assembly 500 is coaxially mounted about the cannula member 114. For example, the expandable balloon assembly 500 may be integrally formed as a single construct. Alternatively, the expandable balloon assembly 500 may be monolithically formed. The expandable balloon assembly 500 may be formed of compressible and/or resilient material. For example, the expandable balloon assembly 500 may be formed of an elastomer. In an aspect, the expandable balloon assembly 500 may be formed of a translucent material. In particular, the expandable balloon assembly 500 includes a plurality of balloons 502 axially arranged along a length of the cannula member 114 in a superposed relation such that the expandable balloon assembly 500 is suitable for use with any tissue thickness. For example, the expandable balloon assembly 500 may have a length greater than half of the length of the cannula member 114. For example, the plurality of balloons 502 may be arranged between proximal and distal portions 114a, 114b of the cannula member 114. In particular, each balloon 502 may be in fluid communication an adjacent balloon 502 such that a single inflation port 138 inflates the entire expandable balloon assembly 500. However, it is contemplated that the expandable balloon assembly 500 may be divided into different inflation zones that are connected to separate inflation ports. For example, distal-most and proximal-most balloons 502 may be inflated by a first inflation source, and the remaining balloons 502 may be inflated by a second inflation source. Each balloon 502 has a toroidal shape. However, the balloons 502 may have various shapes. For example, the balloon 502 may be a fluted balloon. It is contemplated that each balloon may have a different shape such that, e.g., the proximal-most balloon and the distal-most balloon, have different shapes. The expandable balloon assembly 500 expands radially outwardly upon passage of inflation fluids through the fluid port 138. The plurality of balloons 502 may have a uniform thickness and/or diameter to provide uniform expansion when supplied with the inflation fluid. However, based on the amount of inflation fluid supplied to the expandable balloon assembly 500, the plurality of balloons 502 in a sealing relation with tissue may have different diameters. For example, the plurality of balloons 502 may define an hourglass shape (FIG. 2) when engaged with tissue. It is contemplated that each balloon 502 may have a different thickness and/or a diameter to provide different expansion relative to other balloons 502 to enhance contact with tissue.

With reference to FIG. 2, initially, an incision is made in a body wall to gain entry to a body cavity, such as the abdominal cavity. The distal portion 114b of the cannula member 114 of the balloon cannula 100 is inserted through the incision until at least the distal-most balloon 502 of the expandable balloon assembly 500 is within the body cavity. The expandable balloon assembly 500 is inflated after the cannula member 114 is properly placed through the body wall and into the body cavity. A fluid source such as e.g., a syringe (not shown), may be connected to the inflation port 138 to supply an inflation fluid to the expandable balloon assembly 500. The expandable balloon assembly 500 is inflated until the balloons 502 are in a sealing relation with the body wall. At this time, the inflated balloons 502 engaging tissue may have different diameters. With the incision sealed, the body cavity may be insufflated with CO₂, a similar gas, or another insufflation fluid. Surgical instruments (not shown) may be inserted through the balloon cannula 100 to perform desired surgical procedures. To deflate the expandable balloon assembly 500 for removal of the balloon cannula 100 from the body cavity, the inflation port 138 may be opened to release the fluid therethrough. In this manner, the balloon cannula 500 may effectively form a fluid-tight seal against tissue irrespective of the thickness of tissue.

It is further contemplated that the balloon cannula 100 may be used with an obturator (not shown). The obturator generally includes a head portion having latches configured to engage respective notches defined in the cannula housing 112 of the balloon cannula 100 to enhance securement therewith, an elongate shaft extending from the head portion, and an optical penetrating tip coupled to a distal end of the elongate shaft. The optical penetrating tip may be used to penetrate the skin and access the body cavity. By applying pressure against the proximal end of the obturator, the tip of the obturator is forced though the skin and the underlying tissue layers until the cannula and obturator enter the body cavity.

Persons skilled in the art will understand that the devices and methods specifically described herein and illustrated in the accompanying drawings are non-limiting. It is envisioned that the elements and features may be combined with the elements and features of another without departing from the scope of the disclosure. As well, one skilled in the art will appreciate further features and advantages of the disclosure.

What is claimed is:

1. A balloon cannula comprising:
a housing defining a cavity;
a cannula member extending from the housing and defining a lumen in communication with the cavity of the housing;
an expandable balloon assembly disposed along the cannula member, the expandable balloon assembly including a plurality of balloons in a superposed relation, each balloon of the plurality of balloons being in fluid communication with an adjacent balloon of the plurality of balloons; and
a fluid port adapted to be coupled to a fluid source, the fluid port in fluid communication with the expandable balloon assembly.

2. The balloon cannula according to claim 1, wherein the expandable balloon assembly is coaxially mounted about the cannula member.

3. The balloon cannula according to claim 1, wherein the expandable balloon assembly is formed of a compressible material.

4. The balloon cannula according to claim 1, wherein the expandable balloon assembly is integrally or monolithically formed.

5. The balloon cannula according to claim 1, wherein the expandable balloon assembly has a uniform diameter.

6. The balloon cannula according to claim 1, wherein the expandable balloon assembly has a uniform thickness.

7. The balloon cannula according to claim 1, wherein each balloon of the plurality of balloons is configured to expand radially outwards.

8. The balloon cannula according to claim 1, wherein each balloon of the plurality of balloons has a toroidal shape.

9. The balloon cannula according to claim 1, wherein first and second balloons of the plurality of balloons have different thicknesses.

10. The balloon cannula according to claim 1, wherein first and second balloons are radially compressible.

11. The balloon cannula according to claim 1, wherein the plurality of the balloons extends proximally from a distal portion of the cannula member.

12. The balloon cannula according to claim 1, wherein the cannula member has a tapered distal end.

13. The balloon cannula according to claim 1, wherein the cannula member may be formed of a translucent material.

14. The balloon cannula according to claim 1, wherein the expandable balloon assembly is formed of an elastomer.

15. The balloon cannula according to claim 1, wherein the expandable balloon assembly extends between proximal and distal ends of the cannula member.

16. A balloon cannula comprising:
a cannula member defining a lumen dimensioned to receive a surgical object therethrough;
an expandable balloon assembly mounted about the cannula member, the expandable balloon assembly is configured to engage tissue in a sealing relation, the expandable balloon assembly including a plurality of balloons axially arranged between proximal and distal ends of the cannula member; and
a fluid port adapted to be coupled to a fluid source, the fluid port in fluid communication with the expandable balloon assembly.

17. The balloon cannula according to claim 16, wherein each balloon of the plurality of balloons is expandable radially outwards.

18. The balloon cannula according to claim 16, wherein each balloon of the plurality of balloons is formed of a resilient material.

19. The balloon cannula according to claim 16, wherein the plurality of balloons is disposed in a superposed relation.

20. The balloon cannula according to claim 16, wherein the plurality of balloons has a uniform diameter.
